(19) United States
(12) Patent Application Publication
(10) Pub. No.: US 2012/0330205 A1
(43) Pub. Date: Dec. 27, 2012

(54) Posture correcting device

(76) Inventor: Chung Min Liao, Tauyuan (TW)

(21) Appl. No.: 13/134,921

(22) Filed: Jun. 21, 2011

(51) Int. Cl. A61F 5/00

(52) U.S. Cl. 602/19

(57) ABSTRACT

A posture correcting device includes a frame member having a column for engaging with a back portion of a user and having a predetermined shape for forcedly correcting a human posture of the back portion of the user, a beam laterally supported on top of the column and having a pair of curved hooks extended from the lateral beam for engaging with the shoulder portion of the user, and the frame member includes a bar laterally supported on bottom of the column and having an inner portion for engaging with a waist portion of the user, and the frame member includes a pair of straps attached to the lateral bar for engaging onto the waist portion of the user.

### BACKGROUND OF THE INVENTION

1. Field of the Invention

The present invention relates to a posture correcting device, and more particularly to a posture correcting device including a formed or shaped structure or shape for forcedly modifying a human posture or for properly promoting or correcting or modifying or maintaining proper posture when he is slouching his shoulder forward and including a simplified structure for allowing the posture correcting device to be easily and quickly assembled with fewer manufacturing procedures and to be manufactured with a decreased manufacturing cost.

2. Description of the Prior Art

Typical posture correcting devices for forcedly modifying a human posture comprise shoulder, pelvic, knee and foot supports which are corrected to each other by loading elements each of which is embodied in the form of an inextensible strip and an avoid of residual deformation elastic rod fixed thereto.

For example, U.S. Pat. No. 7,744,511 to Grigoriev et al. discloses one of the typical posture correcting devices comprising a support arranged on the thoracic surface of the body which is connected to the shoulder supports with the aid of self-fixing buckles, thereby forming a vest which tightly embraces a patient body.

However, the typical posture correcting device comprises a complicated structure that may not be easily and quickly assembled with fewer manufacturing procedures and that may not be manufactured with a decreased manufacturing cost.

U.S. Pat. No. 7,871,388 to Brown, U.S. Pat. No. 7,901,371 to Vayntraub, and U.S. Pat. No. 7,914,473 to Josey disclose several other typical posture correcting devices each comprising a back plate or garment for engaging with the back portion of the user, and a pair of shoulder pads or tension straps for contacting the front side of the user's shoulder.

However, the typical posture correcting device comprises a pair of shoulder pads or tension straps that are flexible and may not be used for forcedly or properly promoting or correcting or modifying or maintaining the posture of the user.

The present invention has arisen to mitigate and/or obviate the afore-described disadvantages of the conventional posture correcting devices.

### SUMMARY OF THE INVENTION

The primary objective of the present invention is to provide a posture correcting device including a formed or shaped structure or shape for forcedly modifying a human posture or for properly promoting or correcting or modifying or maintaining proper posture when he is slouching his shoulder forward and including a simplified structure for allowing the posture correcting device to be easily and quickly assembled with fewer manufacturing procedures and to be manufactured with a decreased manufacturing cost.

In accordance with one aspect of the invention, there is provided a posture correcting device comprising a frame member including a column having an inner portion for engaging with a back portion of a user and having a predetermined shape for forcedly correcting and maintaining a human posture of the back portion of the user, and the frame member including a lateral beam laterally supported on top of the column and having an inner portion for engaging with a shoulder portion of the user and having a predetermined shape for forcedly correcting and maintaining a human posture of the shoulder portion of the user, and the frame member including a pair of curved hooks extended forwardly from the lateral beam for engaging with the shoulder portion of the user, and the frame member including a lateral bar laterally supported on bottom of the column and having an inner portion for engaging with a waist portion of the user, and the frame member including a pair of straps attached to the lateral bar for engaging onto the waist portion of the user.

The straps are engageable with a fastening device for solidly and stably anchoring or securing or retaining the frame member to the user.

A casing may further be provided and attached onto the frame member. The column and the lateral beam and the lateral bar are engaged into the casing, and the curved hooks are extended out of the casing for engaging with the user's shoulder, and the tension straps are extended out of the casing for engaging with the waist portion of the user.

Further objectives and advantages of the present invention will become apparent from a careful reading of the detailed description provided hereinbelow, with appropriate reference to the accompanying drawings.

### BRIEF DESCRIPTION OF THE DRAWINGS

FIG. 1 is a perspective view of a posture correcting device in accordance with the present invention;

FIG. 2 is a side plan schematic view of the posture correcting device;

FIG. 3 is a perspective view of a skeleton or primary body of the posture correcting device;

FIGS. 4, 5, 6 are cross sectional views of the posture correcting device, taken along lines 4-4, 5-5, 6-6 of FIG. 3 respectively; and

FIG. 7 is a side plan schematic view similar to FIG. 2, illustrating the operation of the posture correcting device.

### DETAILED DESCRIPTION OF THE PREFERRED EMBODIMENT

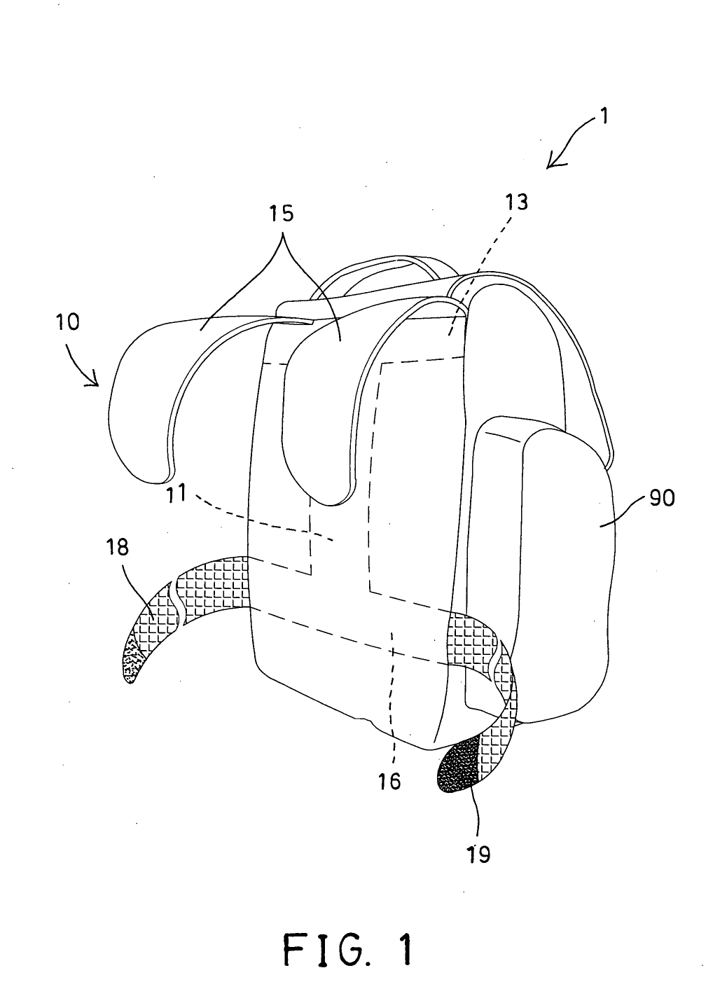

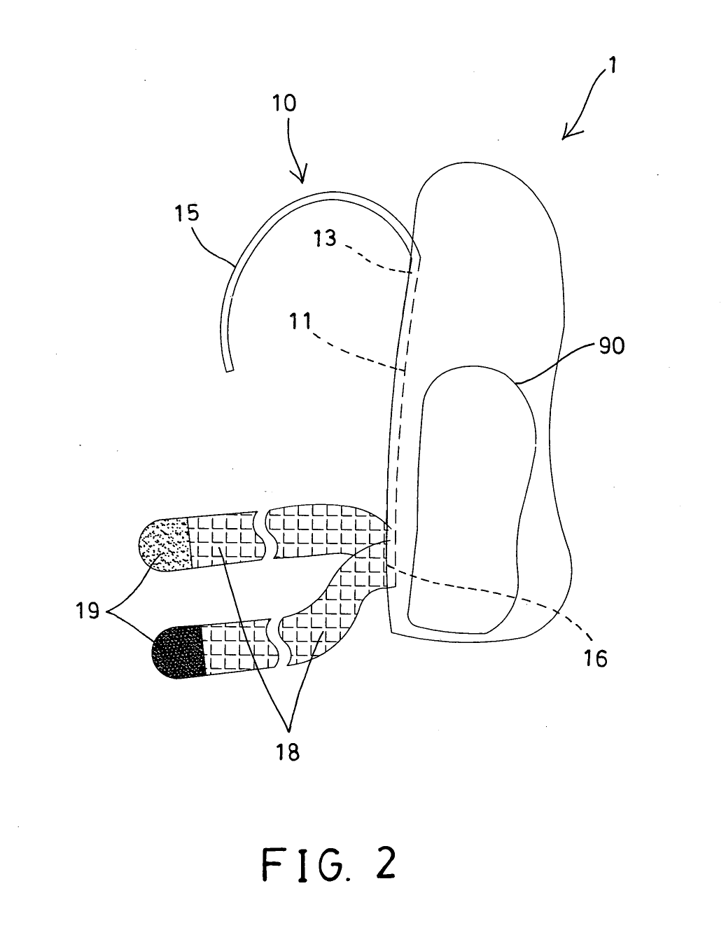

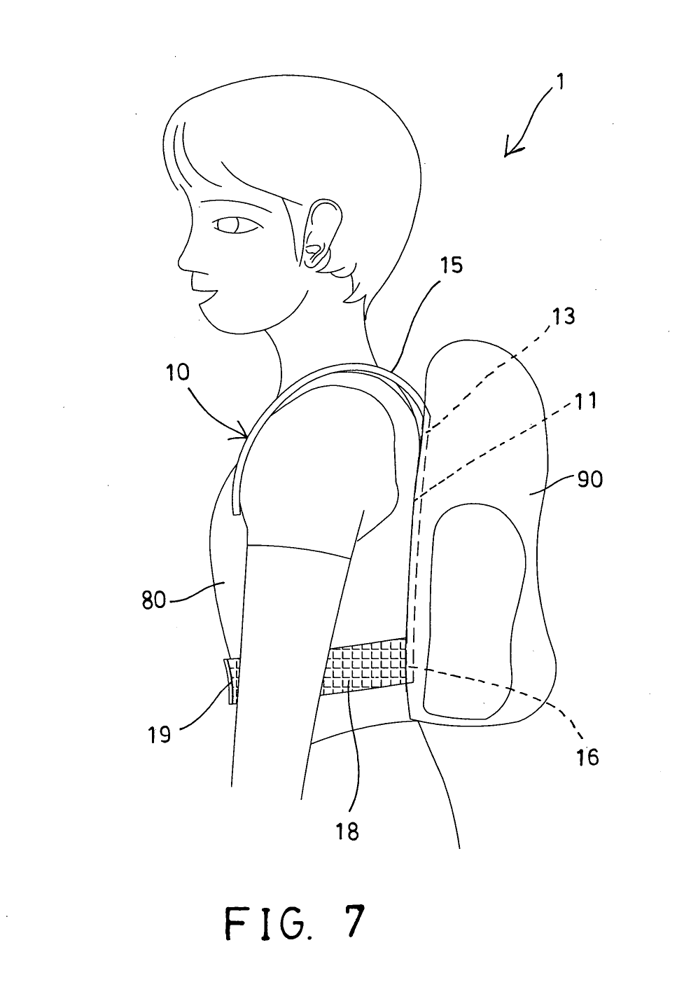

Referring to the drawings, and initially to FIGS. 1 and 2, a posture correcting device 1 in accordance with the present invention comprises a skeleton or primary body or frame member 10 for attaching or mounting or engaging onto the user 80, particularly the chest or shoulder portion of the user 80 (FIG. 7), and a bag or container or luggage case or casing 90 attached or mounted or secured onto the skeleton or primary body or frame member 10 for receiving and carrying various objects (not shown).

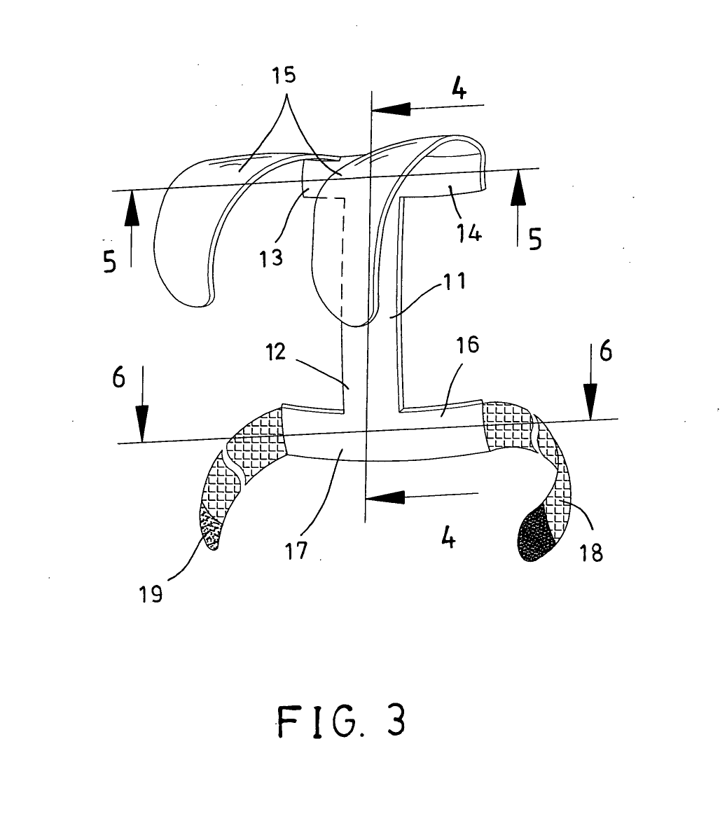

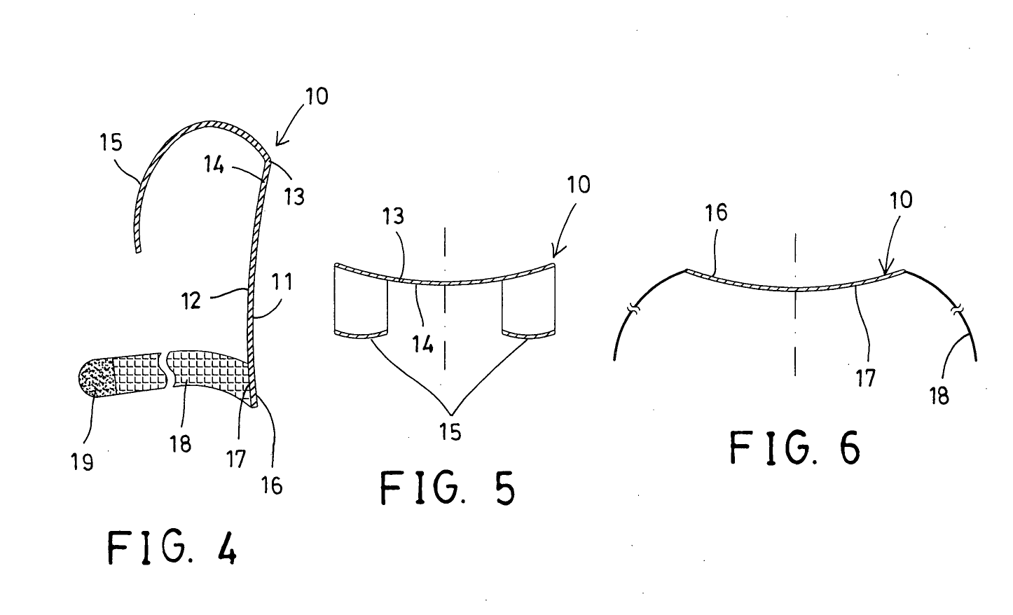

As shown in FIGS. 3-6, the frame member 10 includes a central column 11 having an inner surface or portion 12 for contacting or engaging with the spinal or back portion of the user, and the column 11 is preferably made of strong or stiff materials, such as metal, plastic or thermo-plastic materials which include a predetermined or fixed shape or structure for forcedly promoting or correcting or modifying or maintaining the human posture of the spinal or back portion of the user 80 (FIG. 7).

The frame member 10 further includes a lateral beam 13 laterally disposed or supported on top of the column 11 and having an inner surface or portion 14 for contacting or engaging with the rear side of the user's shoulder, and a pair of curved hooks 15 extended forwardly from the lateral beam 13 for contacting or engaging with the front side and the upper side of the user's shoulder (FIG. 7). Similarly, the lateral beam 13 and the curved hooks 15 are preferably made of strong or stiff materials, such as metal, plastic or thermo-plastic materials which include a predetermined and formed or shaped structure or shape for forcedly promoting or correcting or modifying or maintaining the human posture of the shoulder portion of the user 80 (FIG. 7).

The frame member 10 further includes a lateral bar 16 laterally disposed or supported on bottom of the column 11 and having an inner surface or portion 17 for contacting or engaging with the back or waist portion of the user, and a pair of tension straps 18 attached or mounted or secured to the distal ends of the lateral bar 16 for engaging onto the waist portion of the user 80 (FIG. 7) and for solidly and stably anchoring or securing or retaining the frame member 10 to the user 80 with a fastening device 19, such as a hook and loop fastening device 19. Similarly, the lateral bar 16 is also preferably made of strong or stiff materials, such as metal, plastic or thermo-plastic materials which include a predetermined and formed or shaped structure or shape for forcedly promoting or correcting or modifying or maintaining the human posture of the waist portion of the user 80 (FIG. 7).

As shown in FIGS. 1 and 2, the column 11 and the lateral beam 13 and the lateral bar 16 of the frame member 10 are disposed or received or engaged into the casing 90, and the curved hooks 15 that are extended forwardly from the lateral beam 13 are extended out of the casing 90 for contacting or engaging with the front side and the upper side of the user's shoulder, and the tension straps 18 are also extended out of the casing 90 for contacting or engaging with the waist portion of the user 80 (FIG. 7) and engageable with the fastening device.19 and for solidly and stably anchoring or securing or retaining the frame member 10 to the user 80. The frame member 10 includes a simplified structure that may be easily and quickly assembled with fewer manufacturing procedures and that that may be manufactured with a decreased manufacturing cost.

Accordingly, the posture correcting device in accordance with the present invention includes a formed or shaped structure or shape for forcedly modifying a human posture or for properly promoting or correcting or modifying or maintaining proper posture when he is slouching his shoulder forward and including a simplified structure for allowing the posture correcting device to be easily and quickly assembled with fewer manufacturing procedures and to be manufactured with a decreased manufacturing cost.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A posture correcting device comprising:
a frame member including a column having an inner portion for engaging with a back portion of a user and having a predetermined shape for forcedly correcting and maintaining a human posture of the back portion of the user, and said frame member including a lateral beam laterally supported on top of said column and having an inner portion for engaging with a shoulder portion of the user and having a predetermined shape for forcedly correcting and maintaining a human posture of the shoulder portion of the user, and said frame member including a pair of curved hooks extended forwardly from the lateral beam for engaging with the shoulder portion of the user, and said frame member including a lateral bar laterally supported on bottom of said column and having an inner portion for engaging with a waist portion of the user, and said frame member including a pair of straps attached to said lateral bar for engaging onto the waist portion of the user.

2. The posture correcting device as claimed in claim 1, wherein said straps are engageable with a fastening device.

3. The posture correcting device as claimed in claim 1 further comprising a casing attached onto said frame member.

4. The posture correcting device as claimed in claim 3, wherein said column and said lateral beam and said lateral bar are engaged into said casing, and said curved hooks are extended out of said casing for engaging with the user's shoulder, and said tension straps are extended out of said casing for engaging with the waist portion of the user.
